(19) United States
(12) Patent Application Publication
(10) Pub. No.: US 2016/0351823 A1
(43) Pub. Date: Dec. 1, 2016

(54) BENZOFUROINDOLE DERIVATIVE AND ORGANIC ELECTROLUMINESCENCE DEVICE

(71) Applicant: HODOGAYA CHEMICAL CO., LTD., Tokyo (JP)

(72) Inventors: Makoto NAGAOKA, Tokyo (JP); Kouki KASE, Tokyo (JP); Keigo NAITO, Tokyo (JP); Kanae OTSUKA, Tokyo (JP); Shigeru KUSANO, Tokyo (JP)

(73) Assignee: HODOGAYA CHEMICAL CO., LTD., Tokyo (JP)

(21) Appl. No.: 15/116,639

(22) PCT Filed: Feb. 12, 2015
(86) PCT No.: PCT/JP2015/053752

§ 371 Date: Aug. 4, 2016

(30) Foreign Application Priority Data
Feb. 18, 2014 (JP) 2014-028104

(51) Int. Cl. H01L 51/00; C07D 491/048

(52) U.S. Cl. CPC H01L 51/0071; C07D 491/048; H01L 51/5056

(57) ABSTRACT

According to the present invention, there are provided a benzofuroindole derivative represented by the following general formula (1); and an organic EL device comprising a pair of electrodes and at least one organic layer sandwiched therebetween, wherein the above derivative is used as a constituent material for the at least one organic layer. The benzofuroindole derivative of the present invention is useful as a constituent material for a hole injection layer, a hole transport layer, an electron blocking layer or a luminous layer of an organic EL device. It has excellent electron blocking capability, is stable in a thin film state, and excels in heat resistance. Thus, the organic EL device of the present invention, prepared using such a benzofuroindole derivative, is high in luminous efficiency and power efficiency, thereby lowering the practical driving voltage of the device. The device can also lower light emission starting voltage, and improve durability.

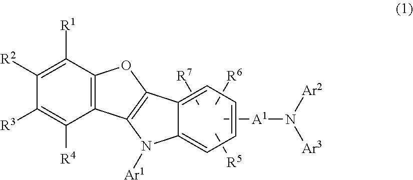

### TECHNICAL FIELD

This invention relates to a compound suitable for an organic electroluminescent device, and the device. More specifically, the invention relates to a benzofuroindole derivative, and an organic electroluminescent device using the derivative.

### BACKGROUND ART

An organic electroluminescent device (may hereinafter be referred to as an organic EL device) is a self light-emitting device, and is thus brighter, better in visibility, and capable of clearer display, than a liquid crystal device. Hence, active researches have been conducted on organic EL devices.

In 1987, C. W. Tang et al. of Eastman Kodak developed a laminated structure device sharing various roles among different materials, thereby imparting practical applicability to organic EL devices using organic materials. They laminated a layer of tris(8-hydroxyquinoline)aluminum (will hereinafter be abbreviated as Alq₃), which is a fluorescent body capable of transporting electrons, and a layer of an aromatic amine compound capable of transporting holes. Upon injecting the charges of electrons and holes into the layer of the fluorescent body to perform light emission, the device was capable of attaining a high luminance of 1,000 cd/m² or more at a voltage of 10V or less (see Patent Document 1 and Patent Document 2).

Many improvements have been made to put the organic EL devices to practical use. For example, high efficiency and durability are achieved by an electroluminescent device sharing the various roles among more types of materials, and having an anode, a hole injection layer, a hole transport layer, a luminous layer, an electron transport layer, an electron injection layer and a cathode provided in sequence on a substrate.

For a further increase in the luminous efficiency, it has been attempted to utilize triplet excitons, and the utilization of phosphorescent luminous compounds has been considered. Furthermore, devices utilizing light emission by thermally activated delayed fluorescence (TADF) have been developed. An external quantum efficiency of 5.3% has been realized by an device using a thermally activated delayed fluorescence material.

The luminous layer can also be prepared by doping a charge transporting compound, generally called a host material, with a fluorescent compound, a phosphorescent luminous compound, or a material radiating delayed fluorescence. The selection of the organic material in the organic EL device greatly affects the characteristics of the device, such as efficiency and durability.

With the organic EL device, the charges injected from both electrodes recombine in the luminous layer to obtain light emission, and how efficiently the charges of the holes and the electrons are passed on to the luminous layer is of importance. Hole injection properties are enhanced, and electron blocking properties which block the electrons injected from the cathode are enhanced to increase the probability of holes and electrons recombining. Moreover, excitons generated within the luminous layer are confined, whereby a high luminous efficiency can be obtained. Thus, the role of the hole transport material is so important that there has been a desire for a hole transport material having high hole injection properties, allowing marked hole mobility, possessing high electron blocking properties, and having high durability to electrons.

In connection with the life of the device, heat resistance and amorphousness of the material are also important. A material with low heat resistance is thermally decomposed even at a low temperature by heat produced during device driving, and the material deteriorates. In a material with low amorphousness, in particular, crystallization of a thin film occurs in a short time, and the device deteriorates. Thus, high resistance to heat and good amorphousness are required of the material to be used.

As the hole transport materials so far used in organic EL devices, N,N′-diphenyl-N,N′-di(α-naphthyl)benzidine (will hereinafter be called NPD for short) and various aromatic amine derivatives have been known (see Patent Document 1 and Patent Document 2). NPD has satisfactory hole transport capability, but its glass transition point (Tg) is as low as 96° C. Thus, it is poor in heat resistance and, under high temperature conditions, it causes deterioration of device characteristics due to crystallization. Among the aromatic amine derivatives of Patent Document 1 and Patent Document 2 are compounds having excellent hole mobility of 10⁻³ cm²/Vs or more. Since their electron blocking properties are insufficient, however, some of electrons pass through the luminous layer, and an improvement in luminous efficiency cannot be expected. For a further increased efficiency, therefore, a material which has higher electron blocking properties, provides a more stable thin film, and features higher in heat resistance has been desired.

As compounds improved in characteristics such as heat resistance, hole injection properties, and electron blocking properties, arylamine compounds having a substituted furoindole structure or a substituted carbazole structure represented by the following formulas (Compound A and Compound B) have been proposed (see Patent Documents 3 and 4).

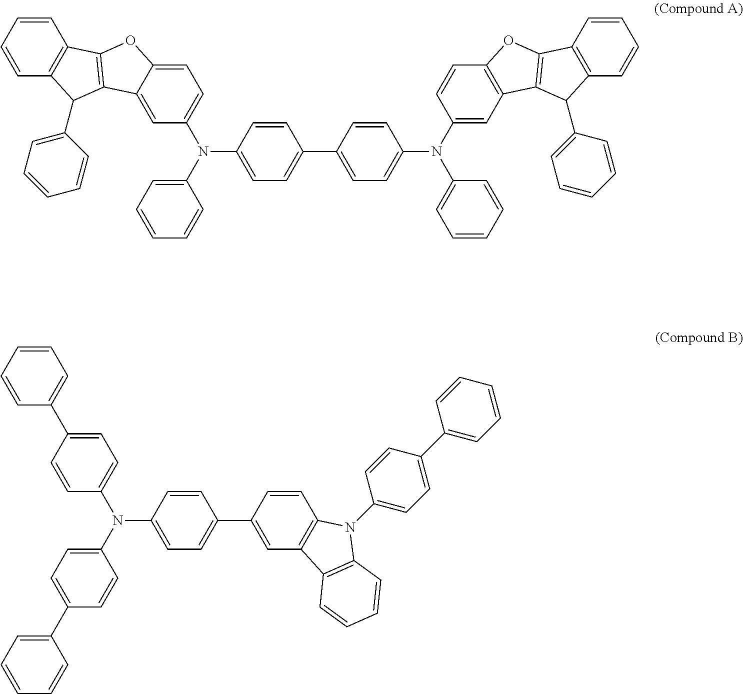 [SMILES: c1ccc(C2c3ccccc3-c3oc4ccc(N(c5ccccc5)c5ccc(-c6ccc(N(c7ccccc7)c7ccc8oc9c(c8c7)C(c7ccccc7)c7ccccc7-9)cc6)cc5)cc4c32)cc1 ; c1ccc(-c2ccc(N(c3ccc(-c4ccccc4)cc3)c3ccc(-c4ccc5c(c4)c4ccccc4n5-c4ccc(-c5ccccc5)cc4)cc3)cc2)cc1]

An device using the above Compound A or Compound B for a hole injection layer or a hole transport layer has been improved in heat resistance, luminous efficiency or the like, but the improvement has been still insufficient. Moreover, current efficiency and lowering of driving voltage have been insufficient, and amorphousness has been problematical. Thus, an even lower driving voltage and an even higher luminous efficiency, with an increase in amorphousness, have been desired.

### PRIOR ART DOCUMENTS

### Patent Documents

Patent Document 1: JP-A-Hei 8-48656
Patent Document 2: Japanese Patent No. 3194657
Patent Document 3: JP-A-2010-205815
Patent Document 4: WO2008/62636

### Non-Patent Documents

Non-Patent Document 1: J. Org. Chem., 60, 7508 (1995)
Non-Patent Document 2: Chem. Rev., 95, 2457 (1995)

### SUMMARY OF THE INVENTION

### Problems to be Solved by the Invention

It is an object of the present invention to provide an organic compound, which is excellent in hole injection/transport performance, has electron blocking capability, is highly stable in a thin film state, and excels in heat resistance, as a material for a high efficiency, high durability organic EL device.

It is another object of the present invention to provide an organic EL device having high efficiency and high durability with the use of this compound.

### Means for Solving the Problems

To attain the above objects, the present inventors noted that an aromatic tertiary amine structure had high ability to inject and transport holes, and expected that a benzofuroindole ring structure would be able to show electron blocking properties, heat resistance, and thin film stability. Against such a background, they designed and chemically synthesized a compound having a benzofuroindole ring structure. Using this compound, moreover, they experimentally produced various organic EL devices, and extensively evaluated the characteristics of the devices. As a result, they have accomplished the present invention.

According to the present invention, there is provided a benzofuroindole derivative represented by the following general formula (1)

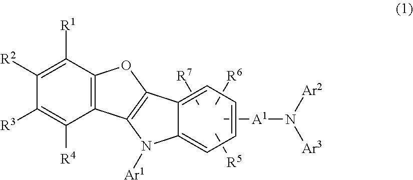

wherein,
Ar¹, Ar² and Ar³ may be the same or different, and each represent an aromatic hydrocarbon group, an aromatic heterocyclic group or a condensed polycyclic aromatic group,
R¹ to R⁷ may be the same or different, each represent a hydrogen atom, a deuterium atom, a fluorine atom, a chlorine atom, a cyano group, a nitro group, an alkyl group having 1 to 6 carbon atoms, a cycloalkyl group having 5 to 10 carbon atoms, an alkenyl group having 2 to 6 carbon atoms, an alkyloxy group having 1 to 6 carbon atoms, a cycloalkyloxy group having 5 to 10 carbon atoms, an aromatic hydrocarbon group, an aromatic heterocyclic group, a condensed polycyclic aromatic group or an aryloxy group, and may be bonded to each other via a single bond, a methylene group, an oxygen atom or a sulfur atom to form a ring,
A¹ represents a divalent group of an aromatic hydrocarbon, a divalent group of an aromatic heterocycle, a divalent group of a condensed polycyclic aromatic or a single bond,
Ar² and Ar³ may be bonded to each other via a single bond, a methylene group, an oxygen atom or a sulfur atom to form a ring, and when A¹ is a divalent group of an aromatic hydrocarbon, a divalent group of an aromatic heterocycle or a divalent group of a condensed polycyclic aromatic,
A¹ and Ar³ may be bonded to each other via a single bond, a methylene group, an oxygen atom or a sulfur atom to form a ring.

For the benzofuroindole derivative of the present invention, the following are preferred:

(A) The benzofuroindole derivative is a benzofuroindole derivative represented by the following general formula (2);

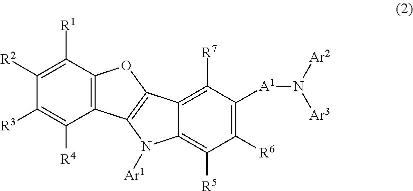

wherein,
Ar¹ to Ar³, R¹ to R⁷, and A¹ have the same meanings as those defined for the aforementioned general formula (1).
   (B) A¹ is a phenylene group.

According to the present invention, moreover, there is provided an organic electroluminescent device comprising a pair of electrodes and at least one organic layer sandwiched therebetween, wherein the benzofuroindole derivative represented by the aforementioned general formula (1) is used as a constituent material for the at least one organic layer.

In the organic electroluminescent device of the present invention, it is preferred that the organic layer be a hole transport layer, an electron blocking layer, a hole injection layer or a luminous layer.

### Effects of the Invention

The benzofuroindole derivative of the present invention is a novel compound, and has the following physical properties:
(1) Hole injection characteristics are satisfactory.
(2) Hole mobility is high.
(3) Electron blocking capability is better than conventional hole transport materials.
(4) Thin film state is more stable than conventional hole transport materials.
(5) Heat resistance is excellent.

Moreover, the organic EL device of the present invention has the following properties:
(6) Luminous efficiency and power efficiency are high.
(7) Light emission starting voltage is low.
(8) Practical driving voltage is low.
(9) Durability is excellent.

The benzofuroindole derivative of the present invention is higher in the hole injection properties, hole mobility, electron blocking properties, and stability to electrons, than conventional materials. With a hole injection layer and/or a hole transport layer prepared using the benzofuroindole derivative of the present invention, therefore, excitons generated within a luminous layer can be confined, and the probability of recombination of holes and electrons can be further increased to obtain a high luminous efficiency. Also, the driving voltage is lowered to enhance the durability of the resulting organic EL device.

The benzofuroindole derivative of the present invention has excellent ability to block electrons, is better in hole transporting properties than conventional materials, and is highly stable in a thin film state. Thus, an electron blocking layer prepared using the benzofuroindole derivative of the present invention has a high luminous efficiency, is lowered in driving voltage, and is improved in current resistance, so that the maximum light emitting brightness of the organic EL device is increased.

The benzofuroindole derivative of the present invention has excellent hole transporting properties, and has a wide bandgap, as compared with conventional materials. Therefore, the benzofuroindole derivative of the present invention are used as a host material to carry a fluorescence emitting substance, a phosphorescence emitting substance or a delayed fluorescence emitting substance, called a dopant, thereon so as to form a luminous layer. This makes it possible to realize the organic EL devise that drives on a decreased voltage and features an increased luminous efficiency.

That is, the benzofuroindole derivative of the present invention is useful as a constituent material for the hole injection layer, the hole transport layer, the electron blocking layer or the luminous layer of an organic EL device. It has excellent electron blocking capability, is stable in a thin film state, and excels in heat resistance. Thus, the organic EL device of the present invention, prepared using such a benzofuroindole derivative, is high in luminous efficiency and power efficiency, thereby making the practical driving voltage of the device low. The device can also lower light emission starting voltage, and improve durability.

### BRIEF DESCRIPTION OF THE DRAWINGS

FIG. 1 is a ¹H-NMR chart diagram of the compound of Example 1 (Compound 7).

FIG. 2 is a ¹H-NMR chart diagram of the compound of Example 2 (Compound 9).

FIG. 3 is a view showing the configuration of the EL devices of Examples 3, 4 and Comparative Example 1.

### MODE FOR CARRYING OUT THE INVENTION

The benzofuroindole derivative of the present invention is a novel compound having a benzofuroindole ring structure, and is represented by the following general formula (1).

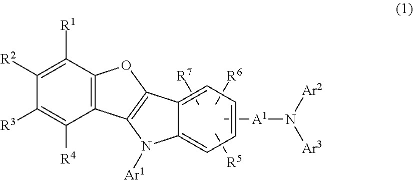

In the benzofuroindole derivative of the present invention, it is preferred that -A¹-N—Ar²Ar³ be bonded at the para-position with respect to the nitrogen atom in the benzene ring of the indole ring. Such a preferred embodiment is represented by the following general formula (2).

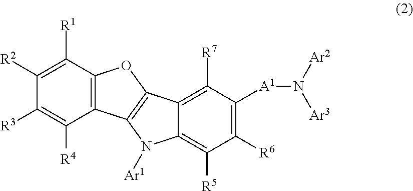

<R¹ to R⁷>

In the general formulas (1) and (2), R¹ to R⁷ may be the same or different, and each represent a hydrogen atom, a deuterium atom, a fluorine atom, a chlorine atom, a cyano group, a nitro group, an alkyl group having 1 to 6 carbon atoms, a cycloalkyl group having 5 to 10 carbon atoms, an alkenyl group having 2 to 6 carbon atoms, an alkyloxy group having 1 to 6 carbon atoms, a cycloalkyloxy group having 5 to 10 carbon atoms, an aromatic hydrocarbon group, an aromatic heterocyclic group, a condensed polycyclic aromatic group, or an aryloxy group. Moreover, R¹ to R⁷ may be bonded to each other via a single bond, a substituted or unsubstituted methylene group, an oxygen atom or a sulfur atom to form a ring, but from the viewpoint of imparting better ability to inject and transport holes, it is preferred for them to exist independently of each other and not to form a ring.

The alkyl group having 1 to 6 carbon atoms, the cycloalkyl group having 5 to 10 carbon atoms or the alkenyl group having 2 to 6 carbon atoms, represented by R¹ to R⁷, can be exemplified by a methyl group, an ethyl group, an n-propyl group, an isopropyl group, an n-butyl group, an isobutyl group, a tert-butyl group, an n-pentyl group, an isopentyl group, a neopentyl group, an n-hexyl group, a cyclopentyl group, a cyclohexyl group, a 1-adamantyl group, a 2-adamantyl group, a vinyl group, an allyl group, an isopropenyl group, a 2-butenyl group and the like. The alkyl group having 1 to 6 carbon atoms, and the alkenyl group having 2 to 6 carbon atoms may be straight-chain or branched.

The alkyl group having 1 to 6 carbon atoms, the cycloalkyl group having 5 to 10 carbon atoms or the alkenyl group having 2 to 6 carbon atoms, represented by R¹ to R⁷, may have a substituent. The substituent can be exemplified by the following, as long as they satisfy a predetermined number of carbon atoms:
a deuterium atom;
a cyano group;
a nitro group;
a halogen atom, for example, a fluorine atom, a chlorine atom, a bromine atom or an iodine atom;
an alkyloxy group having 1 to 6 carbon atoms, for example, a methyloxy group, an ethyloxyl group or a propyloxy group;
an alkenyl group, for example, a vinyl group or an allyl group;
an aryloxy group, for example, a phenyloxy group or a tolyloxy group;
an arylalkyloxy group, for example, a benzyloxy group or a phenethyloxy group;
an aromatic hydrocarbon group or a condensed polycyclic aromatic group, for example, a phenyl group, a biphenylyl group, a terphenylyl group, a naphthyl group, an anthracenyl group, a phenanthrenyl group; a fluorenyl group, an indenyl group, a pyrenyl group, a perylenyl group, a fluoranthenyl group or a triphenylenyl group; and
an aromatic heterocyclic group, for example, a pyridyl group, a thienyl group, a furyl group, a pyrrolyl group, a quinolyl group, an isoquinolyl group, a benzofuranyl group, a benzothienyl group, an indolyl group, a carbazolyl group, a benzoxazolyl group, a benzothiazolyl group, a quinoxalinyl group, a benzimidazolyl group, a pyrazolyl group, a dibenzofuranyl group, a dibenzothienyl group or a carbolinyl group.

Of the above substituents, the alkyloxy group having 1 to 6 carbon atoms may be straight-chain or branched. The above substituents may be further substituted by the above exemplary substituent. Moreover, the substituents may be bonded to each other via a single bond, a substituted or unsubstituted methylene group, an oxygen atom or a sulfur atom to form a ring.

The alkyloxy group having 1 to 6 carbon atoms or the cycloalkyloxy group having 5 to 10 carbon atoms, represented by R¹ to R⁷, can be exemplified by a methyloxy group, an ethyloxy group, an n-propyloxy group, an isopropyloxy group, an n-butyloxy group, a tert-butyloxy group, an n-pentyloxy group, an n-hexyloxy group, a cyclopentyloxy group, a cyclohexyloxy group, a cycloheptyloxy group, a cyclooctyloxy group, a 1-adamantyloxy group, a 2-adamantyloxy group and the like. The alkyloxy group having 1 to 6 carbon atoms may be straight-chain or branched.

The alkyloxy group having 1 to 6 carbon atoms or the cycloalkyloxy group having 5 to 10 carbon atoms, represented by R¹ to R⁷, may have a substituent. The substituent can be exemplified by the same ones as those illustrated as the substituents optionally possessed by the alkyl group having 1 to 6 carbon atoms, the cycloalkyl group having 5 to 10 carbon atoms or the alkenyl group having 2 to 6 carbon atoms, represented by R¹ to R⁷, as long as they satisfy a predetermined number of carbon atoms. Modes which the substituent can adopt are the same as those for the exemplary substituents.

The aromatic hydrocarbon group, the aromatic heterocyclic group or the condensed polycyclic aromatic group, represented by R¹ to R⁷, can be exemplified by a phenyl group, a biphenylyl group, a terphenylyl group, a naphthyl group, an anthracenyl group, a phenanthrenyl group, a fluorenyl group, an indenyl group, a pyrenyl group, a perylenyl group, a fluoranthenyl group, a triphenylenyl group, a pyridyl group, a furyl group, a pyrrolyl group, a thienyl group, a quinolyl group, an isoquinolyl group, a benzofuranyl group, a benzothienyl group, an indolyl group, a carbazolyl group, a benzoxazolyl group, a benzothiazolyl group, a quinoxalinyl group, a benzimidazolyl group, a pyrazolyl group, a dibenzofuranyl group, a dibenzothienyl group, a carbolinyl group and the like. These groups may be bonded to each other via a single bond, a substituted or unsubstituted methylene group, an oxygen atom or a sulfur atom to form a ring.

The aromatic hydrocarbon group, the aromatic heterocyclic group or the condensed polycyclic aromatic group, represented by R¹ to R⁷, may have a substituent. The substituent can be exemplified by the following:
a deuterium atom;
a cyano group;
a nitro group;
a halogen atom, for example, a fluorine atom, a chlorine atom, a bromine atom or an iodine atom;
an alkyl group having 1 to 6 carbon atoms, for example,
a methyl group, an ethyl group, an n-propyl group, an isopropyl group, an n-butyl group, an isobutyl group, a tert-butyl group, an n-pentyl group, an isopentyl group, a neopentyl group or an n-hexyl group;
an alkyloxy group having 1 to 6 carbon atoms, for example, a methyloxy group, an ethyloxyl group or a propyloxy group;
an alkenyl group, for example, a vinyl group or an allyl group;
an aryloxy group, for example, a phenyloxy group or a tolyloxy group;
an arylalkyloxy group, for example, a benzyloxy group or a phenethyloxy group;
an aromatic hydrocarbon group or a condensed polycyclic aromatic group, for example, a phenyl group, a biphenylyl group, a terphenylyl group, a naphthyl group, an anthracenyl group, a phenanthrenyl group, a fluorenyl group, an indenyl group, a pyrenyl group, a perylenyl group, a fluoranthenyl group or a triphenylenyl group;
an aromatic heterocyclic group, for example, a pyridyl group, a thienyl group, a furyl group, a pyrrolyl group, a quinolyl group, an isoquinolyl group, a benzofuranyl group, a benzothienyl group, an indolyl group, a carbazolyl group, a benzoxazolyl group, a benzothiazolyl group, a quinoxalinyl group, a benzimidazolyl group, a pyrazolyl group, a dibenzofuranyl group, a dibenzothienyl group or a carbolinyl group;
an arylvinyl group, for example, a styryl group or a naphthylvinyl group; and
an acyl group, for example, an acetyl group or a benzoyl group.

Of the above substituents, the alkyl group having 1 to 6 carbon atoms and the alkyloxy group having 1 to 6 carbon atoms may be straight-chain or branched. The above substituents may be further substituted by the above substituent. Moreover, the substituents may be bonded to each other via a single bond, a substituted or unsubstituted methylene group, an oxygen atom, or a sulfur atom to form a ring.

The aryloxy group, represented by R¹ to R⁷, can be exemplified by a phenyloxy group, a biphenylyloxy group, a terphenylyloxy group, a naphthyloxy group, an anthracenyloxy group, a phenanthrenyloxy group, a fluorenyloxy group, an indenyloxy group, a pyrenyloxy group, a perylenyloxy group and the like.

The aryloxy group, represented by R¹ to R⁷, may have a substituent. The substituent can be exemplified by the same ones as those illustrated as the substituents optionally possessed by the aromatic hydrocarbon group, the aromatic heterocyclic group or the condensed polycyclic aromatic group represented by R¹ to R⁷. The same holds true of the feasible embodiments for the substituents.

<Ar¹ to Ar³>

In the general formulas (1) and (2), Ar¹ to Ar³ may be the same or different, and each represent an aromatic hydrocarbon group, an aromatic heterocyclic group or a condensed polycyclic aromatic group. The aromatic hydrocarbon group, the aromatic heterocyclic group or the condensed polycyclic aromatic group represented by Ar¹ to Ar³ can be exemplified by the same ones as those illustrated as the aromatic hydrocarbon group, the aromatic heterocyclic group or the condensed polycyclic aromatic group represented by R¹ to R⁷. Ar¹ to Ar³ may be bonded to each other via a single bond, a substituted or unsubstituted methylene group, an oxygen atom, or a sulfur atom to form a ring, but from the viewpoint of imparting better ability to inject and transport holes, it is preferred for them to exist independently of each other and not to form a ring. This is in common with R¹ to R⁷.

The aromatic hydrocarbon group, the aromatic heterocyclic group or the condensed polycyclic aromatic group represented by Ar¹ to Ar³ may have a substituent. The substituent can be exemplified by the same ones as those illustrated as the substituents optionally possessed by the aromatic hydrocarbon group, the aromatic heterocyclic group or the condensed polycyclic aromatic group represented by R¹ to R⁷. The same holds true of the feasible embodiments for the substituents.

### <A¹>

In the above general formulas (1) and (2) A¹ represents a divalent group of an aromatic hydrocarbon, a divalent group of an aromatic heterocycle, a divalent group of a condensed polycyclic aromatic, or a single bond. Examples of the divalent group of the aromatic hydrocarbon, aromatic heterocycle or condensed polycyclic aromatic represented by A¹ are a phenylene group, a biphenylene group, a terphenylene group, a tetrakisphenylene group, a naphthylene group, an anthracenylene group, a phenanthrenylene group, a fluorenylene group, an indenylene group, a pyrenylene group, a perylenylene group, a fluoranthenylene group, a triphenylenylene group, a pyridinylene group, a pyrimidinylene group, a quinolylene group, an isoquinolylene group, an indolylene group, a carbazolylene group, a quinoxalinylene group, a benzimidazolylene group, a pyrazolylene group, a naphthyridinylene group, a phenanthrolinylene group, an acridinylene group, a thienylene group, a benzothienylene group, a benzothiazolylene group, a dibenzothienylene group and the like.

When A¹ is a divalent group of an aromatic hydrocarbon, a divalent group of an aromatic heterocycle, or a divalent group of a condensed polycyclic aromatic, A′ may bind to an aromatic hydrocarbon group, an aromatic heterocyclic group or a condensed polycyclic aromatic group represented by Ar³ via a single bond, a substituted or unsubstituted methylene group, an oxygen atom, or a sulfur atom to form a ring. From the viewpoint of imparting better ability to inject and transport holes, however, it is preferred for them to exist independently of each other and not to form a ring.

The divalent group of the aromatic hydrocarbon, aromatic heterocycle or condensed polycyclic aromatic, represented by Ar¹, may have a substituent. The substituent can be exemplified by the same substituents as those illustrated as the substituents that may be possessed by the aforementioned aromatic hydrocarbon group, aromatic heterocyclic group or condensed polycyclic aromatic group represented by R¹ to R⁷. The same holds true of the feasible embodiments for the substituents.

### PREFERRED EMBODIMENTS

As the benzofuroindole derivative of the present invention, the one represented by the general formula (2) is preferred.

Preferred embodiments of the respective groups in the general formula (1) or (2) are the following from the viewpoint of ease of synthesis:

As R¹ to R⁷, hydrogen, deuterium or an alkyl group having 1 to 6 carbon atoms is preferred, and hydrogen or a lower alkyl group having 1 to 4 carbon atoms is more preferred.

As the aromatic heterocyclic group represented by R¹ to R⁷, a sulfur-containing aromatic heterocyclic group, such as a thienyl group, a benzothienyl group, a benzothiazolyl group or a dibenzothienyl group, is preferred.

As Ar¹ to Ar³, an aromatic hydrocarbon group or a condensed polycyclic aromatic group is preferred, and a phenyl group, a biphenylyl group, or a fluorenyl group is more preferred.

As A¹, a single bond, a divalent group of an aromatic hydrocarbon or a divalent group of a condensed polycyclic aromatic is preferred, and a phenylene group, a biphenylene group or a fluorenylene group is more preferred.

### <Manufacturing Method>

The benzofuroindole derivative of the present invention can be synthesized, for example, by the following manufacturing method: A benzofuroindole derivative having groups corresponding to R¹ to R⁷ which the desired benzofuroindole derivative has is provided, and the 10-position of such a derivative is substituted by an aryl group. Then, its 3-position is brominated using bromine, N-bromosuccinimide or the like. The resulting bromo-substituted product is reacted with pinacolborane, bis(pinacolato)diboron or the like to synthesize a boronic acid or a boronic ester (see, for example, Non-Patent Document 1). The resulting boronic acid or boronic ester is subjected to a cross-coupling reaction (see, for example, Non-Patent Document 2), such as Suzuki coupling, whereby the benzofuroindole derivative of the present invention is synthesized.

The above-mentioned benzofuroindole derivative substituted at the 10-position by an aryl group is brominated to introduce a bromo group at its position other than the 3-position. Then, the resulting product is subjected to the same cross-coupling reaction as above, whereby a benzofuroindole derivative different in the position of substitution can be synthesized.

Alternatively, a benzofuroindole derivative having a bromo group is provided, and its 10-position is substituted by an aryl group in the same manner as above. Then, this product is converted into a boronic acid or a boronic ester, which is then subjected to a cross-coupling reaction such as Suzuki coupling, whereby the benzofuroindole derivative of the present invention can be synthesized.

The purification of the resulting compound can be performed, for example, by purification using a column chromatograph, adsorption purification using silica gel, activated carbon, activated clay, NH silica gel or the like, recrystallization or crystallization using a solvent, sublimation purification and the like. Identification of the compound can be performed by NMR analysis. As physical property values, a glass transition point (Tg) and a work function can be measured.

The glass transition point (Tg) serves as an index to stability in a thin film state. The glass transition point (Tg) can be measured, for example, with a high sensitivity differential scanning calorimeter (DSC3100S, produced by Bruker AXS K.K.) using a powder.

The work function serves as an index to hole transporting properties. The work function can be measured, for example, by preparing a 100 nm thin film on an ITO substrate and using an ionization potential measuring device (PYS-202, produced by Sumitomo Heavy Industries, Ltd.) on the sample.

Of the benzofuroindole derivatives of the present invention, concrete examples of the preferred compounds will be shown below, but the present invention is in no way limited to these compounds. Compounds 1 to 4 are missing.

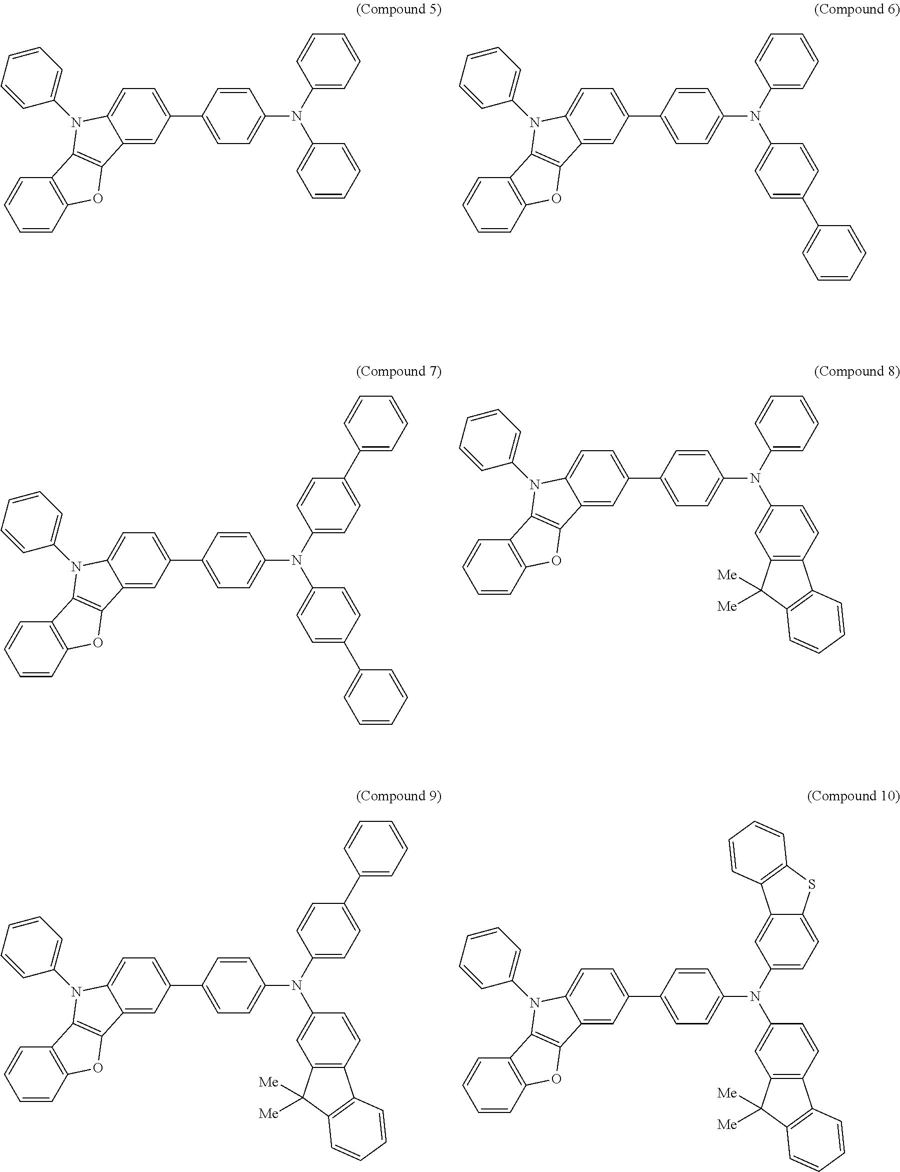

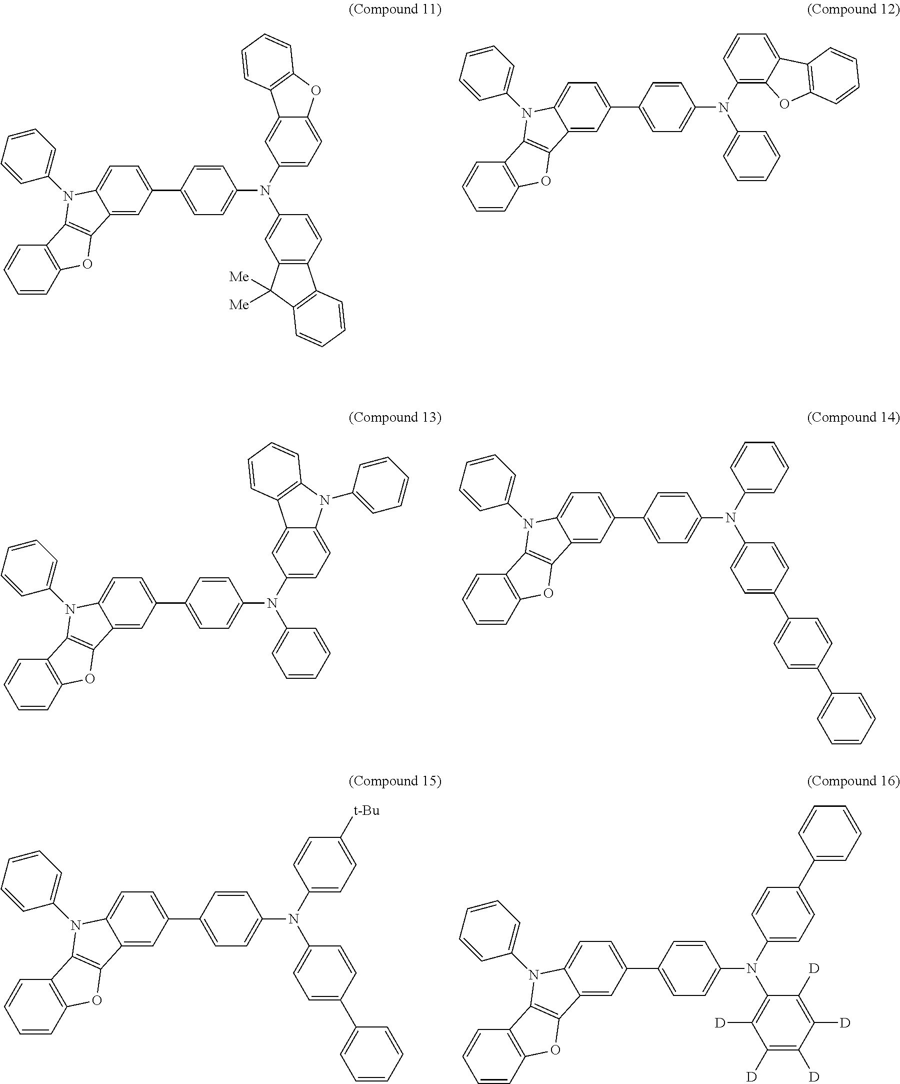

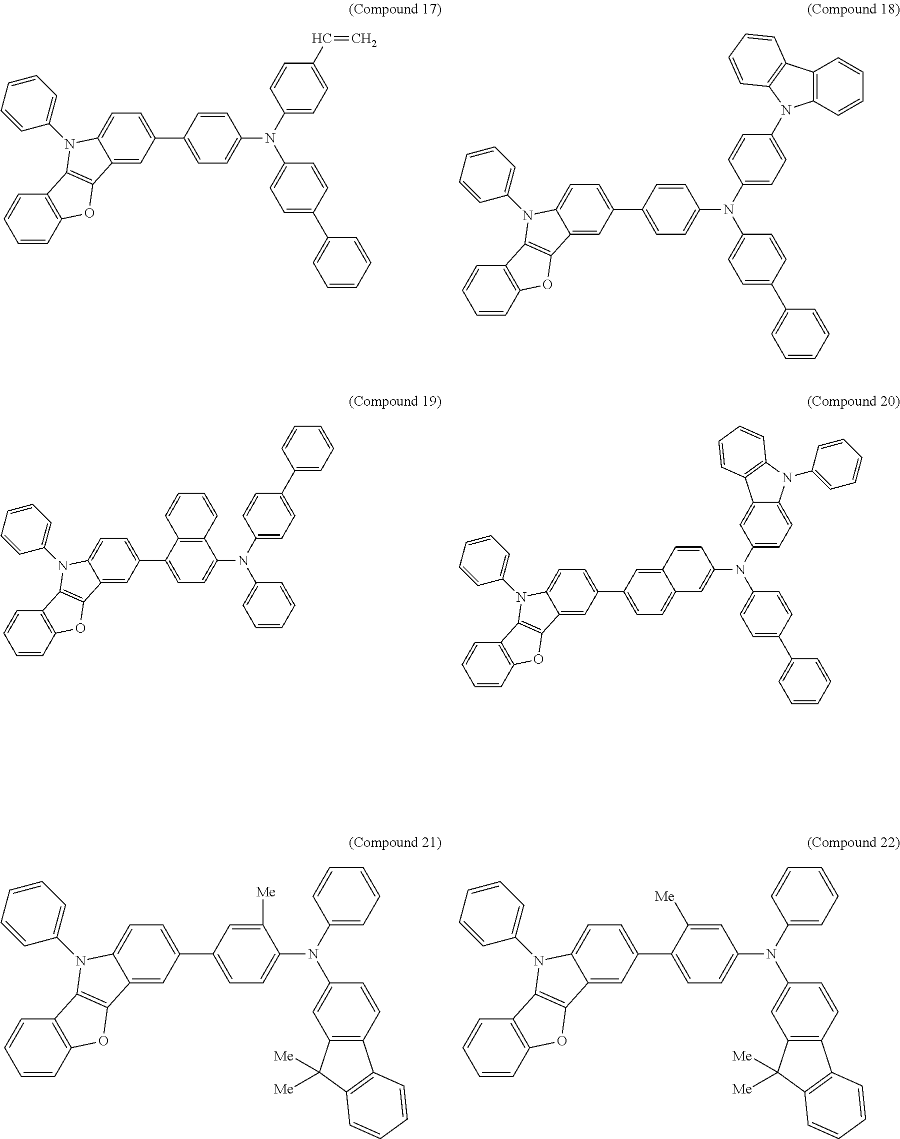

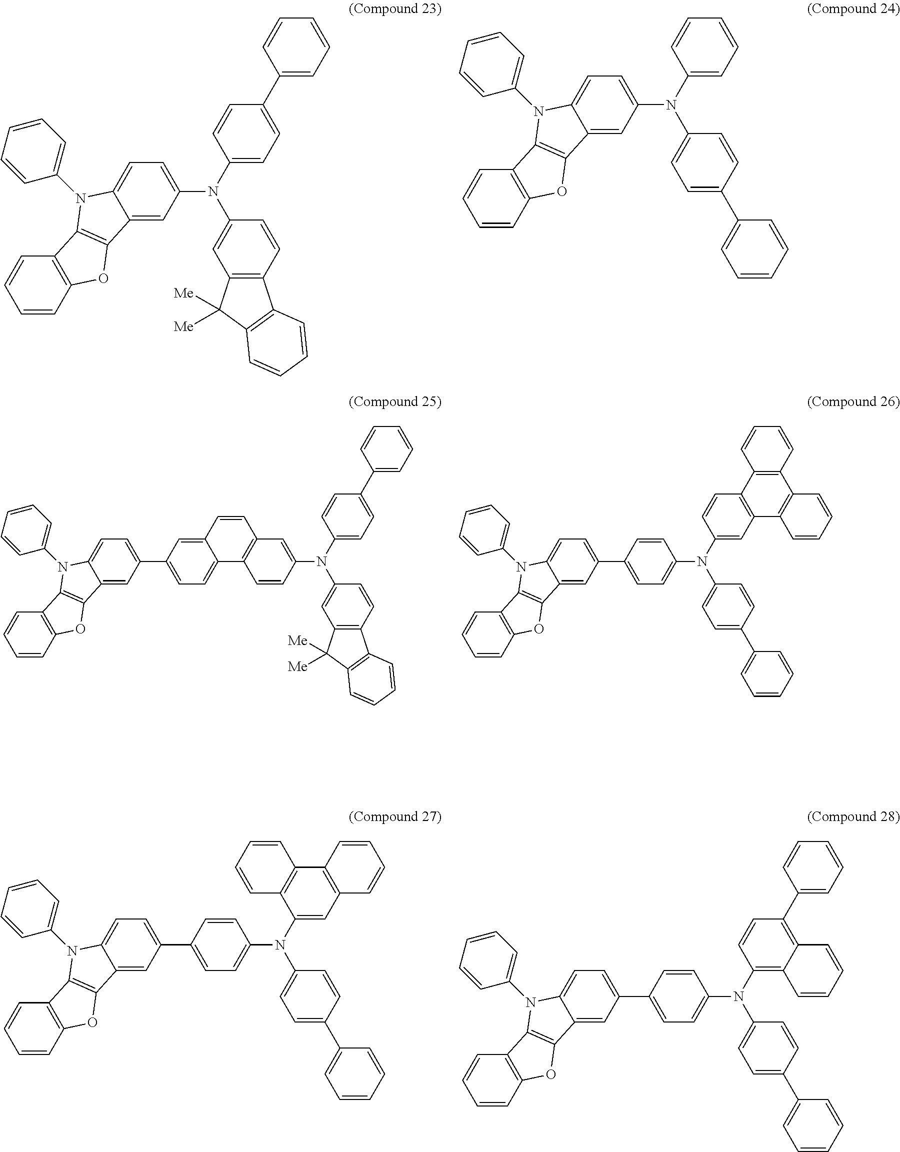

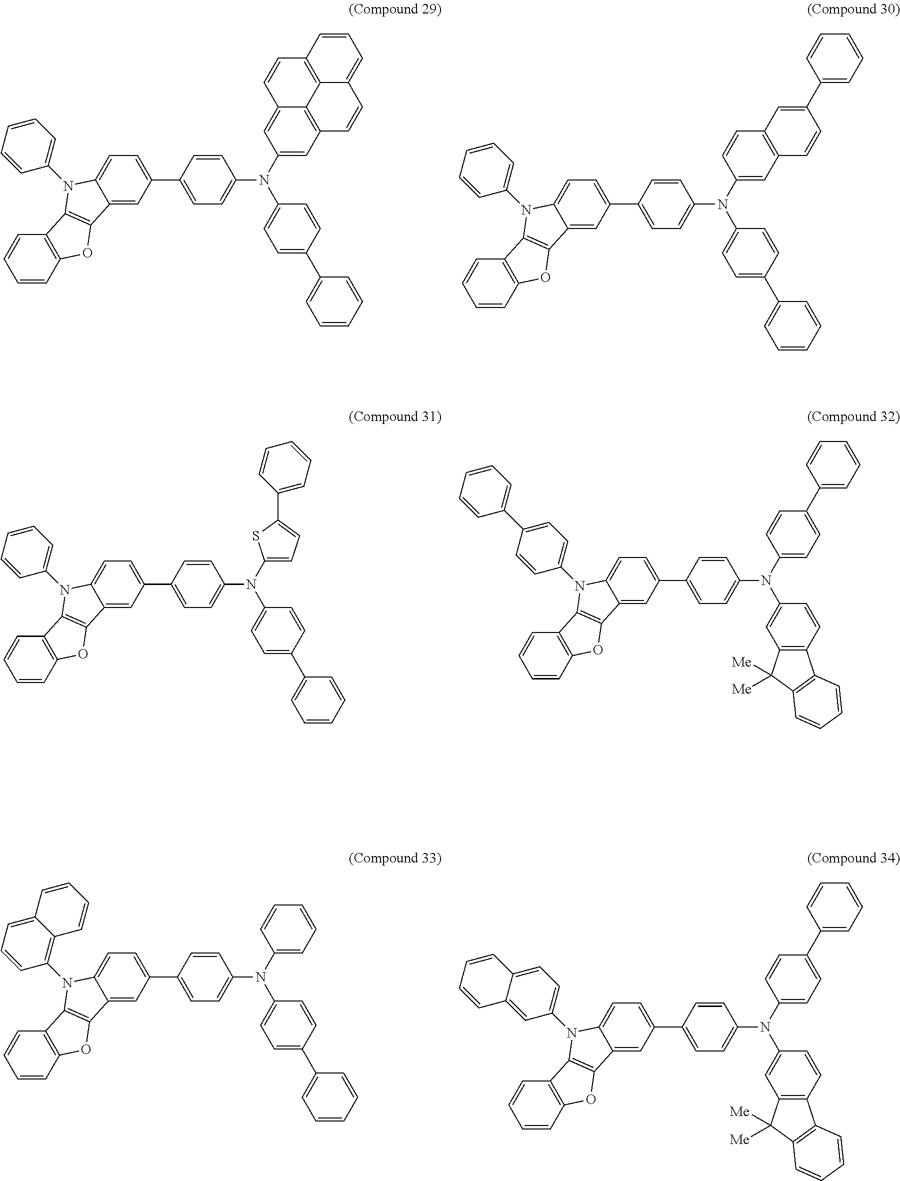

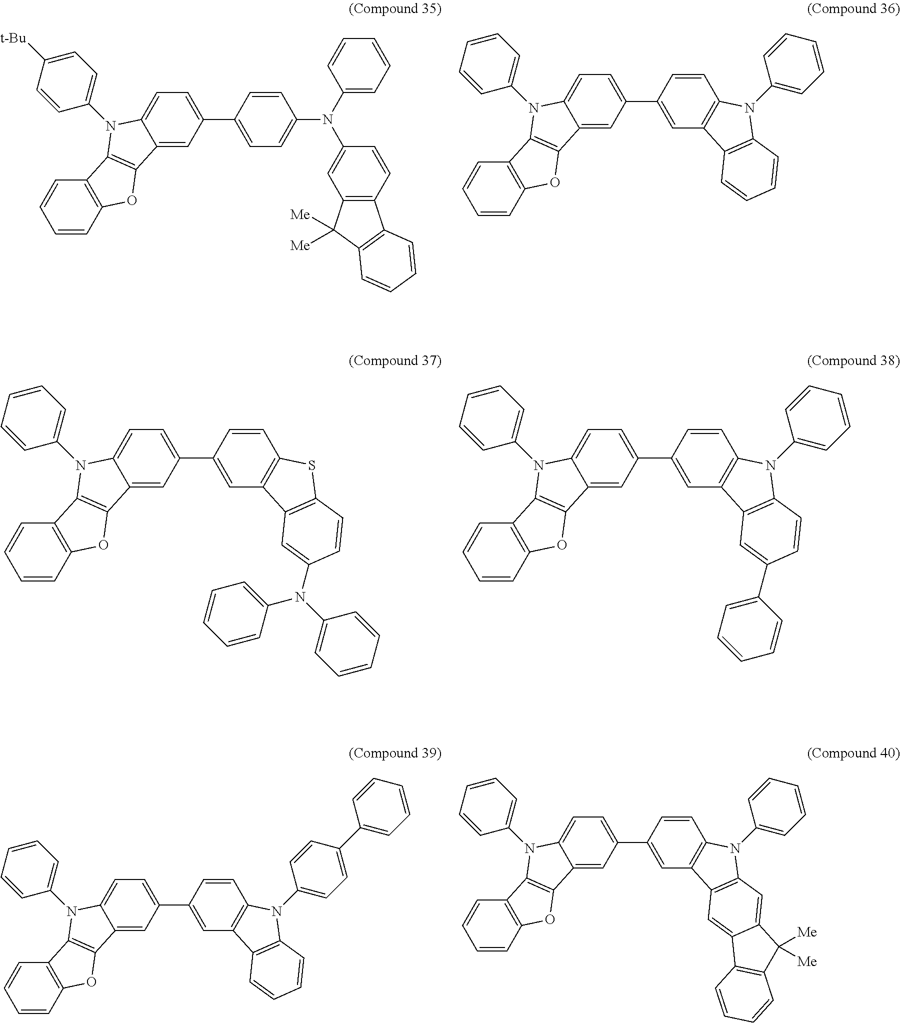 [SMILES: CC(C)(C)c1ccc(-n2c3ccc(-c4ccc(N(c5ccccc5)c5ccc6c(c5)C(C)(C)c5ccccc5-6)cc4)cc3c3oc4ccccc4c32)cc1 ; c1ccc(-n2c3ccccc3c3cc(-c4ccc5c(c4)c4oc6ccccc6c4n5-c4ccccc4)ccc32)cc1 ; c1ccc(N(c2ccccc2)c2ccc3sc4ccc(-c5ccc6c(c5)c5oc7ccccc7c5n6-c5ccccc5)cc4c3c2)cc1 ; c1ccc(-c2ccc3c(c2)c2cc(-c4ccc5c(c4)c4oc6ccccc6c4n5-c4ccccc4)ccc2n3-c2ccccc2)cc1 ; c1ccc(-c2ccc(-n3c4ccccc4c4cc(-c5ccc6c(c5)c5oc7ccccc7c5n6-c5ccccc5)ccc43)cc2)cc1 ; CC1(C)c2ccccc2-c2cc3c4cc(-c5ccc6c(c5)c5oc7ccccc7c5n6-c5ccccc5)ccc4n(-c4ccccc4)c3cc21]

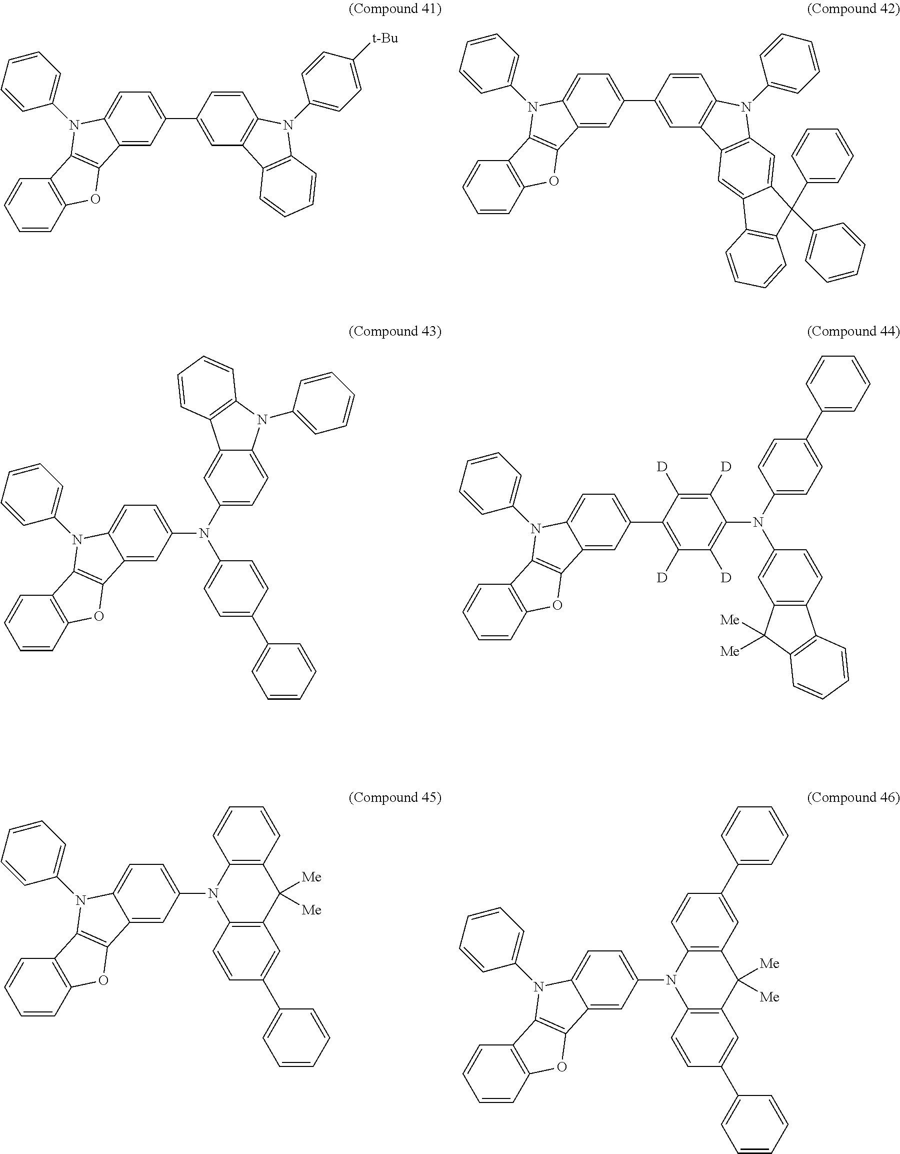

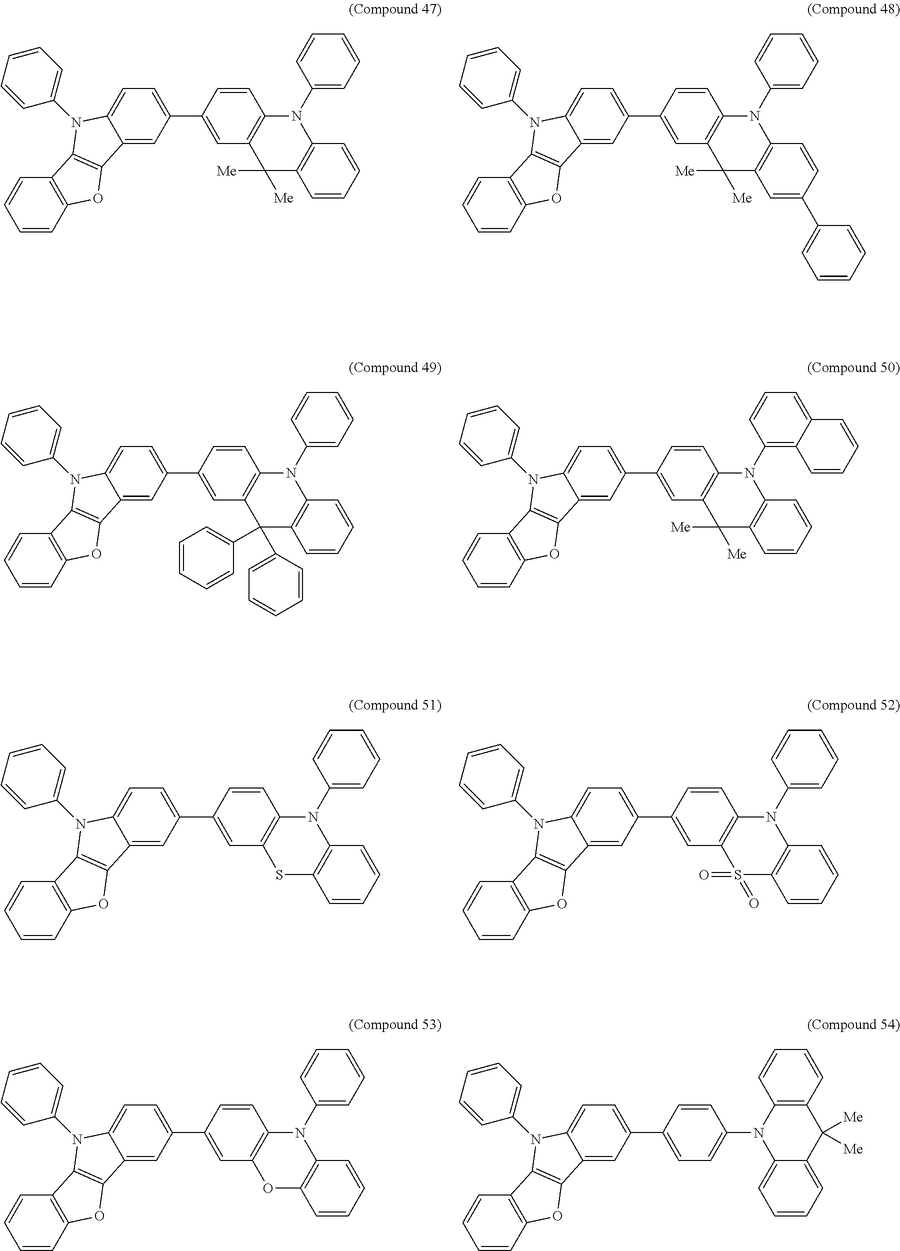

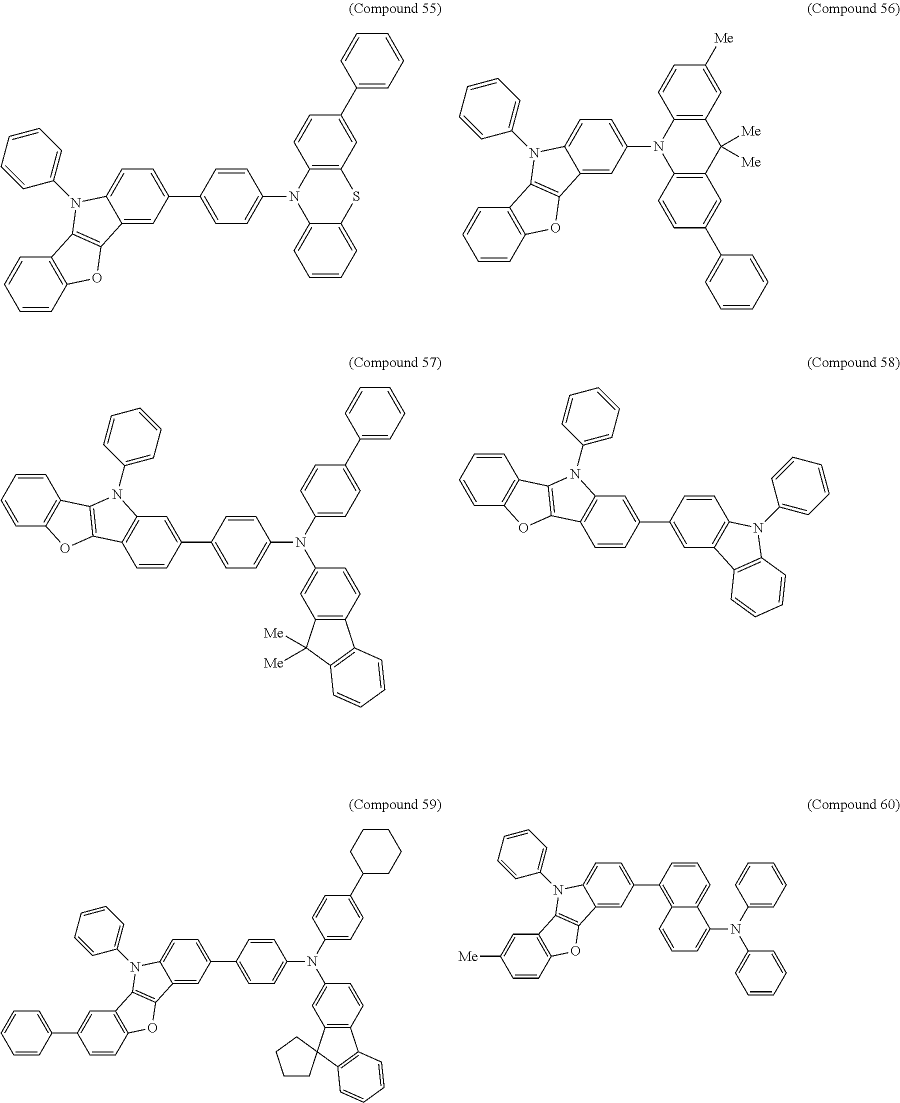

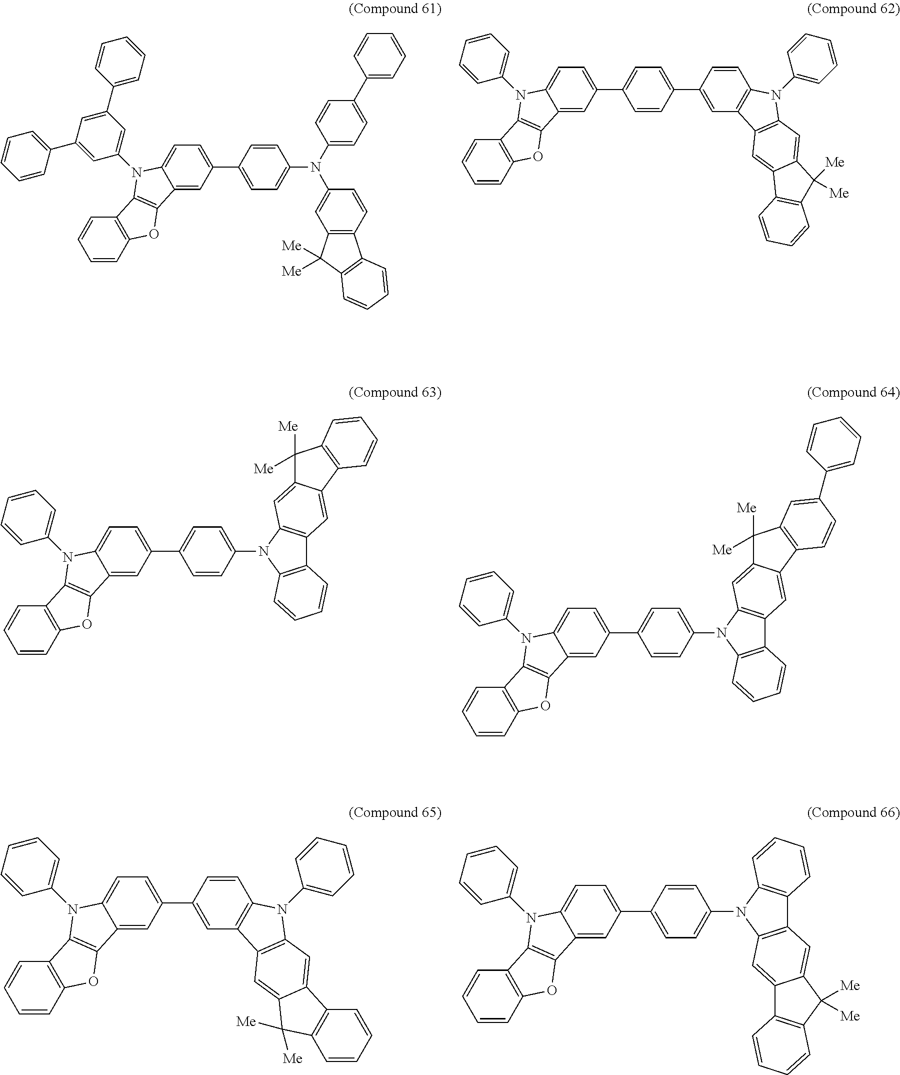

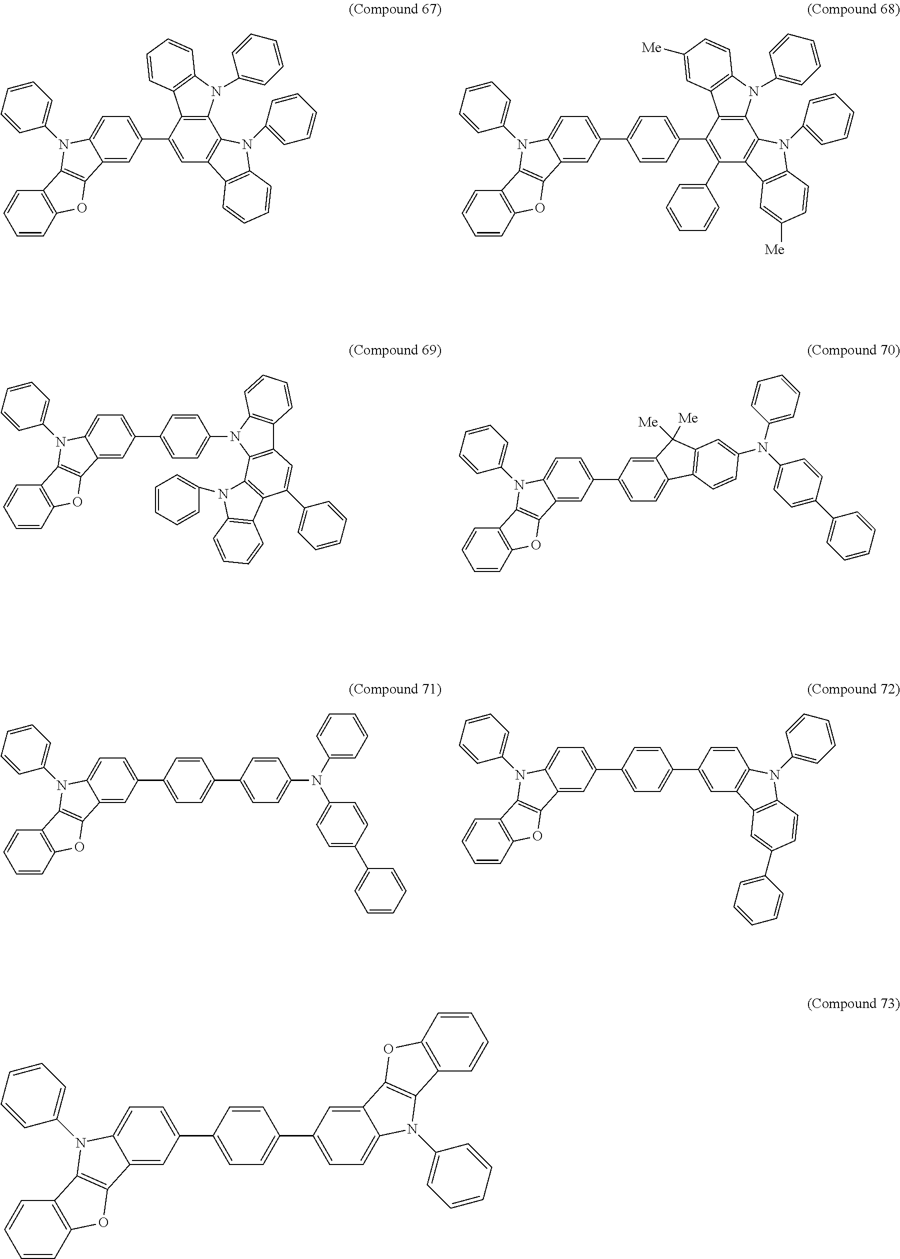

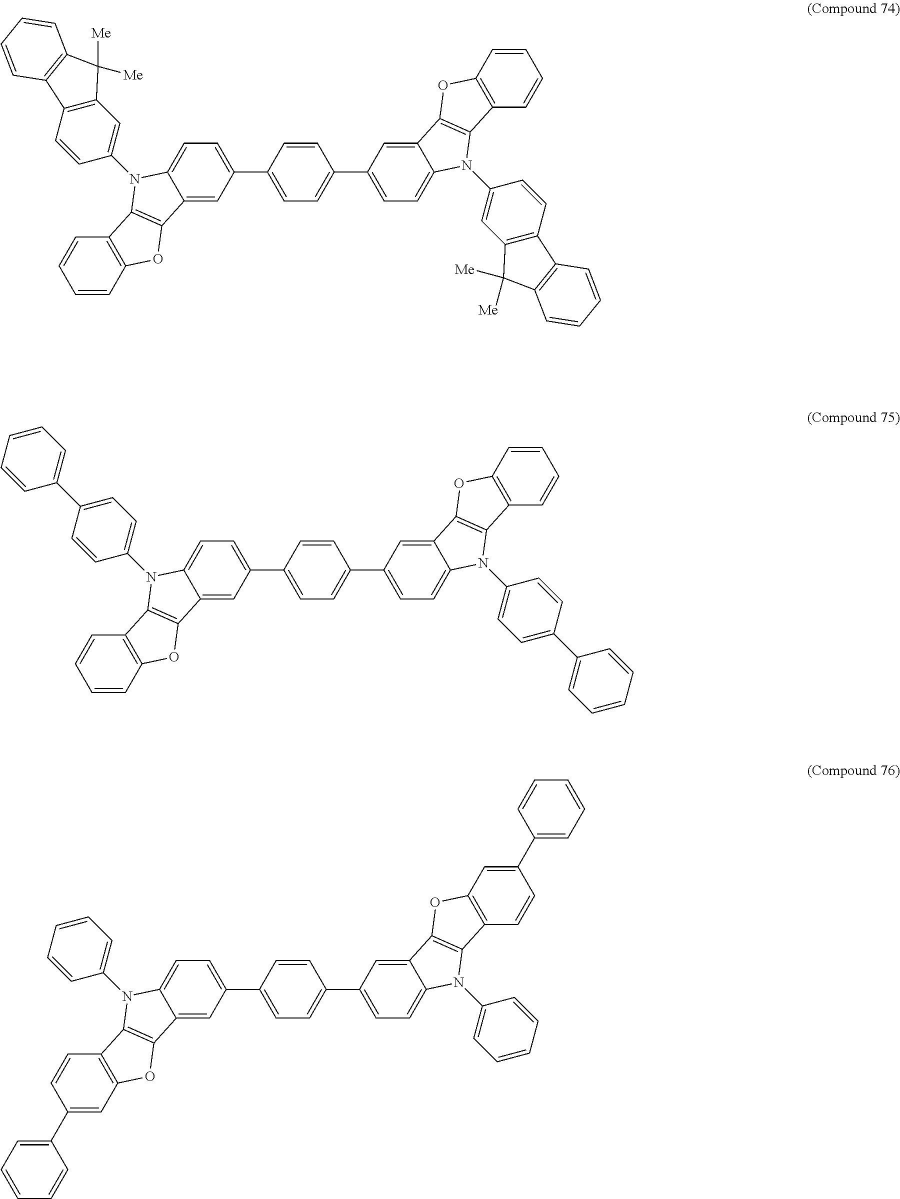 [SMILES: CC1(C)c2ccccc2-c2ccc(-n3c4ccc(-c5ccc(-c6ccc7c(c6)c6oc8ccccc8c6n7-c6ccc7c(c6)C(C)(C)c6ccccc6-7)cc5)cc4c4oc5ccccc5c43)cc21 ; c1ccc(-c2ccc(-n3c4ccc(-c5ccc(-c6ccc7c(c6)c6oc8ccccc8c6n7-c6ccc(-c7ccccc7)cc6)cc5)cc4c4oc5ccccc5c43)cc2)cc1 ; c1ccc(-c2ccc3c(c2)oc2c4cc(-c5ccc(-c6ccc7c(c6)c6oc8cc(-c9ccccc9)ccc8c6n7-c6ccccc6)cc5)ccc4n(-c4ccccc4)c32)cc1]

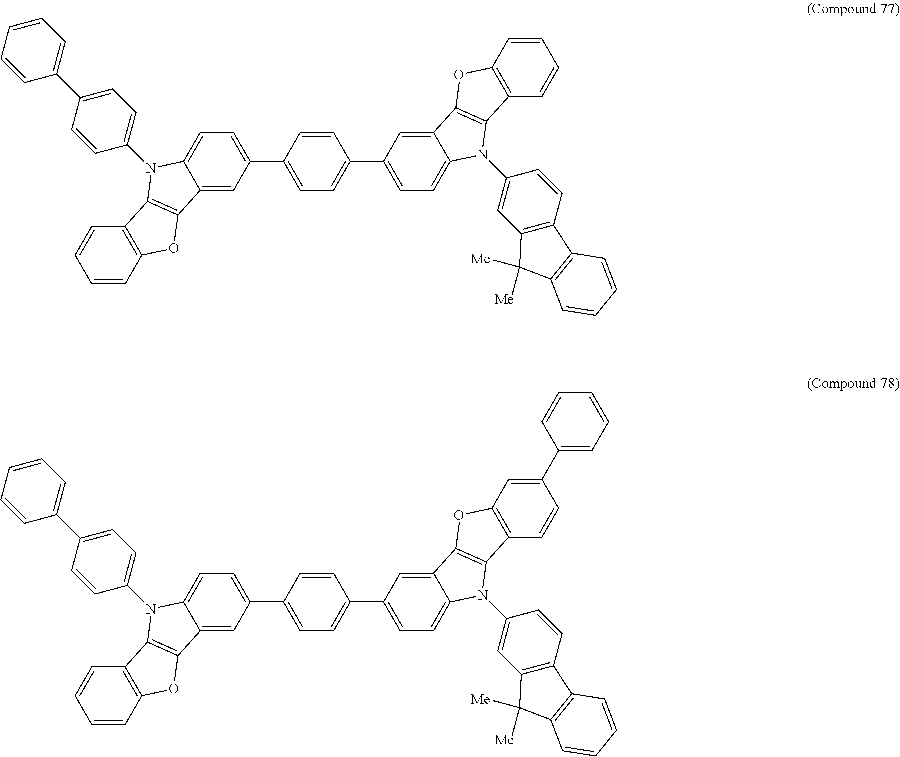 [SMILES: CC1(C)c2ccccc2-c2ccc(-n3c4ccc(-c5ccc(-c6ccc7c(c6)c6oc8ccccc8c6n7-c6ccc(-c7ccccc7)cc6)cc5)cc4c4oc5ccccc5c43)cc21 ; CC1(C)c2ccccc2-c2ccc(-n3c4ccc(-c5ccc(-c6ccc7c(c6)c6oc8ccccc8c6n7-c6ccc(-c7ccccc7)cc6)cc5)cc4c4oc5cc(-c6ccccc6)ccc5c43)cc21]

As will be understood from the above Compounds 73 to 78, the benzofuroindole derivative of the present invention can adopt an embodiment having a molecular structure in which the portion corresponding to the group Ar³ is a benzofuranyl group, and the furan ring in this benzofuranyl group is bound to a benzene ring which is a part of the group A¹, via a single bond. In other words, the benzofuroindole derivative of the present invention can have a symmetric structure composed of two benzofuroindole rings bound together by a connecting group A², if the molecule is viewed as a whole, as represented by the following general formula (3), preferably, the following formula (4):

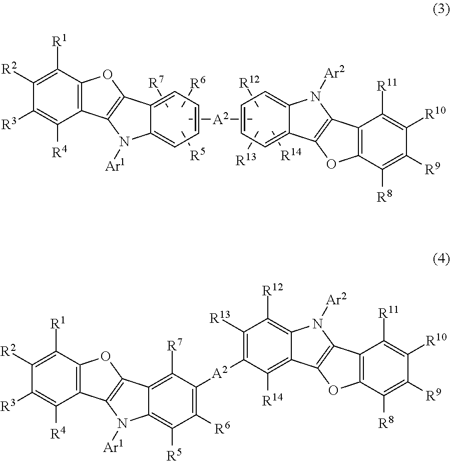

wherein,
Ar¹ to Ar³ and R¹ to R⁷ have the same meanings as those defined in the aforementioned general formula (1),
R⁸ to R¹⁴ have the same meanings as those defined for R¹ to R⁷ in the general formula (1), and
A² stands for a residue remaining after removing a benzene ring, which has been bound via single bonds to both of a nitrogen atom and a furan ring, from a divalent group of an aromatic hydrocarbon, an aromatic heterocycle, or a condensed polycyclic aromatic represented by A¹ in the general formula (1).

### <Organic EL Device>

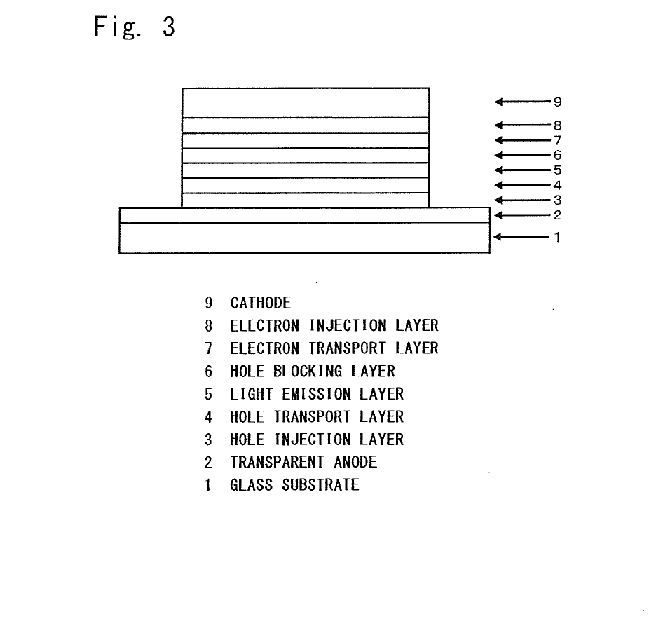

An organic EL device having organic layers formed using the benzofuroindole derivative of the present invention described above (may hereinafter be referred to as the organic EL device of the present invention) has a layered structure, for example, as shown in FIG. 3. That is, in the organic EL device of the present invention, for example, an anode 2, a hole injection layer 3, a hole transport layer 4, a luminous layer 5, a hole blocking layer 6, an electron transport layer 7, an electron injection layer 8 and a cathode 9 are provided in sequence on a substrate 1. The organic EL device of the present invention is not limited to such a structure, but for example, may have an electron blocking layer (not shown) between the hole transport layer 4 and the luminous layer 5. In this multilayer structure, some of the organic layers may be omitted. For example, there can be a configuration in which the hole injection layer 3 between the anode 2 and the hole transport layer 4, the hole blocking layer 6 between the luminous layer 5 and the electron transport layer 7, and the electron injection layer 8 between the electron transport layer 7 and the cathode 9 are omitted, and the anode 2, the hole transport layer 4, the luminous layer 5, the electron transport layer 7 and the cathode 9 are provided sequentially on the substrate 1.

The anode 2 may be composed of an electrode material publicly known per se and, for example, an electrode material having a great work function, such as ITO or gold, is used.

The hole injection layer 3 can be formed using the following material, as well as the benzofuroindole derivative of the present invention:

porphyrin compounds typified by copper phthalocyanine;

triphenylamine derivatives of starburst type;

various triphenylamine tetramers;

acceptor type heterocyclic compounds, for example, hexacyanoazatriphenylene; and

coating type polymeric materials.

The hole injection layer 3 (thin film) can be formed by vapor deposition or any other publicly known method such as a spin coat method or an ink jet method. Various layers to be described below can be similarly formed as films by a publicly known method such as vapor deposition, spin coating or ink jetting.

The hole transport layer 4 can be formed using the following material, as well as the benzofuroindole derivative of the present invention:
benzidine derivatives, for example,
N,N′-diphenyl-N,N′-di(m-tolyl)benzidine (hereinafter abbreviated as TPD),
N,N′-diphenyl-N,N′-di(α-naphthyl)benzidine (hereinafter abbreviated as NPD), and
N,N,N′,N′-tetrabiphenylylbenzidine;
1,1-bis[4-(di-4-tolylamino)phenyl]cyclohexane (hereinafter abbreviated as TAPC); and
various triphenylamine trimers and tetramers.
   The above hole transport materials may be used singly for film formation, but may also be mixed with other materials for film formation. It is permissible to form a plurality of layers with the use of one or more of the above materials, and use a multilayer film composed of a stack of such layers as the hole transport layer.

In the present invention, moreover, it is also possible to forma layer concurrently serving as the hole injection layer 3 and the hole transport layer 4. Such a hole injection/transport layer can be formed using a coating type polymeric material such as poly(3,4-ethylenedioxythiophene) (hereinafter abbreviated as PEDOT)/poly(styrenesulfonate) (hereinafter abbreviated as PSS).

In forming the hole injection layer 3 (hole transport layer 4 as well), materials usually used for forming the layer, materials P-doped with trisbromophenylaminium hexachloroantimonate, or a polymeric compound having the structure of TPD in its partial structure can also be used.

The electron blocking layer (not shown) can be formed using a publicly known compound having an electron blocking action, in addition to the benzofuroindole derivative of the present invention. The publicly known electron blocking compound can be exemplified by the following:
carbazole derivatives, for example,
4,4′,4″-tri(N-carbazolyl)triphenylamine (hereinafter abbreviated as TCTA),
9,9-bis[4-(carbazol-9-yl)phenyl]fluorene,
1,3-bis(carbazol-9-yl)benzene (hereinafter abbreviated as mCP), and
2,2-bis(4-carbazol-9-ylphenyl)adamantane (hereinafter abbreviated as Ad-Cz); and
compounds having a triphenylsilyl group and a triarylamine structure, for example,
9-[4-(carbazol-9-yl)phenyl]-9-[4-(triphenylsilyl) phenyl]-9H-fluorene.
   The above material for the electron blocking layer may be used alone for film formation, but may be mixed with other material for film formation. It is permissible to form a plurality of layers with the use of one or more of the above materials, and use a multilayer film composed of a stack of such layers as the electron blocking layer.

The luminous layer 5 can be formed using a publicly known material. The publicly known material can be exemplified by the following:

metal complexes of quinolinol derivatives including Alq₃;

various metal complexes;

anthracene derivatives;

bisstyrylbenzene derivatives;

pyrene derivatives;

oxazole derivatives; and

polyparaphenylenevinylene derivatives.

The luminous layer 5 may be composed of a host material and a dopant material. As the host material, thiazole derivatives, benzimidazole derivatives and polydialkylfluorene derivatives can be used in addition to the benzofuroindole derivative of the present invention and the above-mentioned luminescent materials.

Usable as the dopant material are, for example, quinacridone, coumarin, rubrene, perylene and derivatives thereof; benzopyran derivatives; rhodamine derivatives; and aminostyryl derivatives.

The luminous layer 5 can also be formed using one or more of the luminescent materials. The luminous layer 5 can be in a single-layer configuration, or can have a multilayer structure composed of a plurality of layers stacked.

Furthermore, a phosphorescent luminous body can be used as the luminescent material. As the phosphorescent luminous body, a luminous phospher in the form of a metal complex containing iridium, platinum or the like can be used.

Concretely, a green luminous phospher such as Ir(ppy)₃; a blue luminous phospher such as FIrpic or FIr6; or a red luminous phospher such as Btp₂Ir(acac); and the like can be used. Any of these luminous phosphers can be used as a dopant for a hole injecting/transporting host material or an electron transporting host material. As the hole injecting/transporting host material, carbazole derivatives, for example, 4,4′-di(N-carbazolyl)biphenyl (hereinafter abbreviated as CBP), TCTA and mCP can be used in addition to the benzofuroindole derivative of the present invention.

Examples of the electron transporting host material are as follows:
p-bis(triphenylsilyl)benzene (hereinafter abbreviated as UGH2); and
2,2′,2″-(1,3,5-phenylene)-tris(1-phenyl-1H-benzimidazole) (hereinafter abbreviated as TPBI).

By using any such material, a high performance organic EL device can be prepared.

The host material is desirably doped with the phosphorescent luminous material in an amount in a range of 1 to 30% by weight relative to the whole luminous layer relying on the vacuum coevaporation in order to avoid concentration quenching.

A material which emits delayed fluorescence, such as a CDCB derivative, for example, PIC-TRZ, CC2TA, PXZ-TRZ, 4CzIPN and the like, can be used as the luminescent material.

The hole blocking layer 6 can be formed using a publicly known compound having hole blocking properties. The publicly known compound having the hole blocking properties can be exemplified by the following:
phenanthroline derivatives, for example, bathocuproine (hereinafter abbreviated as BCP);
metal complexes of quinolinol derivatives, for example, aluminum (III)
bis(2-methyl-8-quinolinato)-4-phenylphenolate (hereinafter abbreviated as BAlq);
various rare earth complexes;
triazole derivatives;
triazine derivatives; and
oxadiazole derivatives.
   The hole blocking layer 6 can also have a single-layer structure or a multilayer laminated structure, and each layer is formed using one or more of the aforementioned compounds having hole blocking action.

The above-mentioned publicly known material having the hole blocking properties can also be used for the formation of the electron transport layer 7 to be described blow. That is, the layer concurrently serving as the hole blocking layer 6 and the electron transport layer 7 can be formed by using the above-mentioned publicly known material having the hole blocking properties.

The electron transport layer 7 is formed using a publicly known compound having electron transporting properties. The publicly known compound having the electron transporting properties can be exemplified by the following:
metal complexes of quinolinol derivatives including Alq₃ and BAlq;
various metal complexes;
triazole derivatives;
triazine derivatives;
oxadiazole derivatives;
thiadiazole derivatives;
carbodiimide derivatives;
quinoxaline derivatives;
phenanthroline derivatives; and
silole derivatives.
   The electron transport layer can also have a single-layer structure or a multilayer laminated structure, and each layer is formed using one or more of the aforementioned compounds having electron transporting action.

The electron injection layer 8 can be formed using a compound publicly known per se, examples of which are as follows:
alkali metal salts such as lithium fluoride and cesium fluoride;
alkaline earth metal salts such as magnesium fluoride; and
metal oxides such as aluminum oxide.
   Upon preferred selection of the electron transport layer and the cathode, the electron injection layer 8 can be omitted.

In connection with the cathode 9, an electrode material with a low work function such as aluminum, or an alloy having a lower work function, such as a magnesium-silver alloy, a magnesium-indium alloy or an aluminum-magnesium alloy, is used as an electrode material.

### EXAMPLES

The present invention will be described more concretely by way of Examples, but the present invention is in no way limited to the following Examples.

### Example 1

### Synthesis of Compound 7

### Synthesis of bis(biphenyl-4-yl)-{4-(10-phenyl-10H-benzo[4,5]furo[3,2-b]indol-3-yl)phenyl}amine

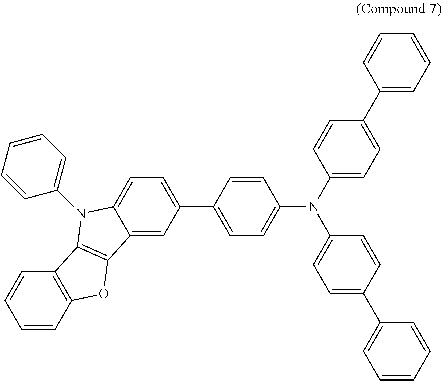 [SMILES: c1ccc(-c2ccc(N(c3ccc(-c4ccccc4)cc3)c3ccc(-c4ccc5c(c4)c4oc6ccccc6c4n5-c4ccccc4)cc3)cc2)cc1]

In a nitrogen atmosphere, a reaction vessel was charged with
3-bromo-10-phenyl-10H-benzo[4,5]furo[3,2-b]indole 5.0 g,
bis(biphenyl-4-yl)-{4-(4,4,5,5-tetramethyl-[1,3,2]dioxaboran-2-yl)phenyl}amine 8.0 g,
mixed solution of toluene/ethanol (4/1, v/v) 100 ml and
2M aqueous solution of potassium carbonate 20 ml.
   Under ultrasonic irradiation, a nitrogen gas was passed through the mixture for 30 minutes. To the mixture, tetrakis(triphenylphosphine)palladium (0.8 g) was added, and the system was heated, and stirred for 6.5 hours at 70° C. The mixture was cooled to room temperature, and then an organic layer was collected by liquid separation. The organic layer was dehydrated over anhydrous magnesium sulfate, and then concentrated under reduced pressure to obtain a crude product. Toluene was added to the crude product to dissolve it, whereafter NH silica gel was added, and adsorption purification was carried out. An inorganic residue was separated by filtration, and the filtrate was concentrated to obtain 5.3 g of bis(biphenyl-4-yl)-{4-(10-phenyl-10H-benzo[4,5]furo[3,2-b]indol-3-yl)phenyl}amine (Compound 7) as a white powder (yield 56.6%).

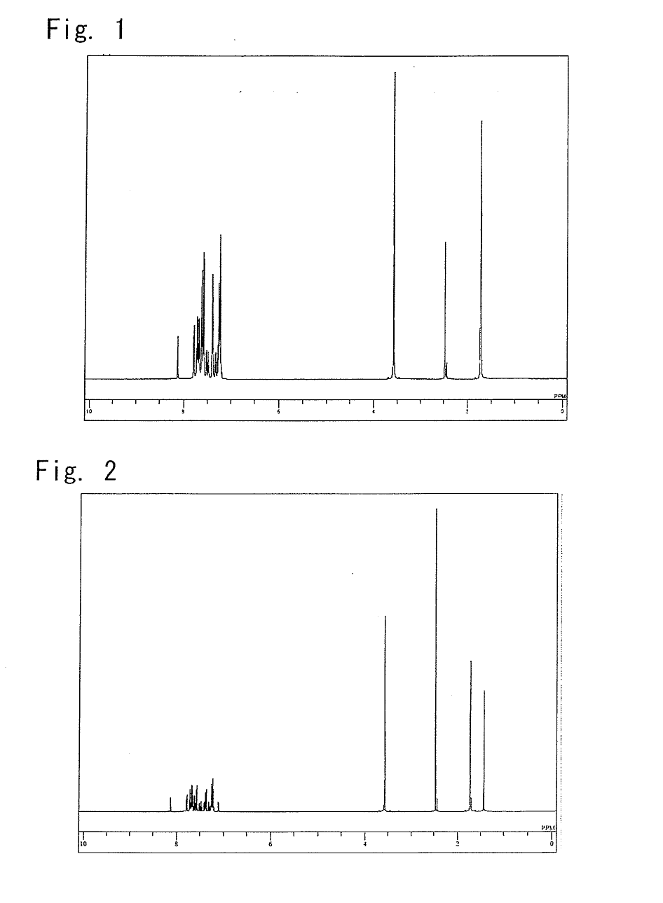

In connection with the resulting white powder, its structure was identified using NMR. The results of its ¹H-NMR measurement are shown in FIG. 1. In ¹H-NMR (THF-d₈), the following signals of 34 hydrogens were detected:

δ (ppm)=8.13 (1H)
7.79 (2H)
7.74-7.65 (6H)
7.64-7.56 (9H)
7.52 (1H)
7.48 (1H)
7.39 (4H)
7.33 (1H)
7.29-7.21 (9H)

### Example 2

### Synthesis of Compound 9

### Synthesis of (biphenyl-4-yl)-(9,9-dimethyl-9H-fluoren-2-yl)-{4-(10-phenyl-10H-benzo[4,5]furo[3,2-b]indol-3-yl)phenyl}amine

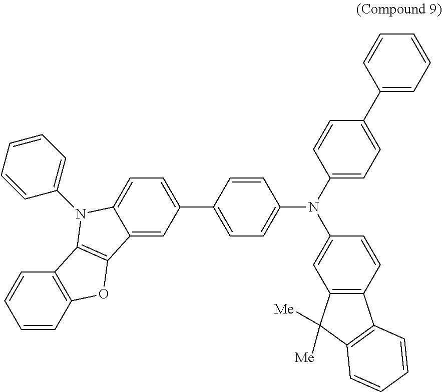 [SMILES: CC1(C)c2ccccc2-c2ccc(N(c3ccc(-c4ccccc4)cc3)c3ccc(-c4ccc5c(c4)c4oc6ccccc6c4n5-c4ccccc4)cc3)cc21]

In a nitrogen atmosphere, a reaction vessel was charged with

3-bromo-10-phenyl-10H-benzo[4,5]furo[3,2-b]indole 5.0 g,

(biphenyl-4-yl)-(9,9-dimethyl-9H-fluoren-2-yl)-{4-(4,4,5,5-tetramethyl-[1,3,2]dioxaboran-2-yl)phenyl}amine 8.6 g,

mixed solution of toluene/ethanol (4/1, v/v) 100 ml and

2M aqueous solution of potassium carbonate 20 ml.

Under ultrasonic irradiation, a nitrogen gas was passed through the mixture for 30 minutes. To the mixture, tetrakis(triphenylphosphine)palladium (0.8 g) was added, and the system was heated, and stirred for 8.5 hours at 70° C. After the mixture was cooled to room temperature, an organic layer was collected by liquid separation. The organic layer was dehydrated over anhydrous magnesium sulfate, and then concentrated under reduced pressure to obtain a crude product. The crude product was purified by column chromatography (carrier: silica gel, eluent: toluene/n-hexane), and then subjected to crystallization purification using a toluene/methanol mixed solution to obtain 2.6 g (yield 26.2%) of (biphenyl-4-yl)-(9,9-dimethyl-9H-fluoren-2-yl)-{4-(10-phenyl-10H-benzo[4,5]furo[3,2-b]indol-3-yl)phenyl}amine (Compound 9) as a light yellow powder.

In connection with the resulting light yellow powder, its structure was identified using NMR. The results of its ¹H-NMR measurement are shown in FIG. 2. In ¹H-NMR (THF-d₈), the following signals of 38 hydrogens were detected:

δ (ppm)=8.13 (1H)
7.79 (2H)
7.74-7.65 (8H)
7.63 (2H)
7.60 (1H)
7.57 (2H)
7.52 (1H)
7.48 (1H)
7.43-7.37 (4H)
7.33 (1H)
7.29-7.20 (8H)
7.12 (1H)

The benzofuroindole derivatives of the present invention obtained in the foregoing Examples were measured for the glass transition point by a high sensitivity differential scanning calorimeter (DSC3100S, produced by Bruker AXS K.K.).

| | Glass transition point |
|---|---|
| Compound of Example 1 | 125.6° C. |
| Compound of Example 2 | 134.3° C. |

The compounds of the present invention have a glass transition point of 100° C. or higher, particularly, 120° C. or higher, demonstrating that the compounds of the present invention are stable in a thin film state.

### <Measurement of Work Function>

Using each of the benzofuroindole derivatives of the present invention obtained in the above Examples, a vapor deposited film with a film thickness of 100 nm was prepared on an ITO substrate, and its work function was measured using an ionization potential measuring device (PYS-202, produced by Sumitomo Heavy Industries, Ltd.).

| | Work function |
|---|---|
| Compound of Example 1 | 5.64 eV |
| Compound of Example 2 | 5.59 eV |
| NPD(HTM-A) | 5.54 eV |

The benzofuroindole derivatives of the present invention showed an energy level superior to a work function of 5.5 eV shown by a general hole transport material such as NPD or TPD, and are thus found to have satisfactory hole transport capability.

### Evaluation of organic EL device characteristics

### Example 3

A hole injection layer 3, a hole transport layer 4 (using Compound 7 obtained in Example 1), a luminous layer 5, a hole blocking layer 6, an electron transport layer 7, an electron injection layer 8 and a cathode (aluminum electrode) 9 were vapor deposited in this order on an ITO electrode formed beforehand as a transparent anode 2 on a glass substrate 1 to prepare an organic EL device as shown in FIG. 3.

Concretely, the glass substrate 1 having a 50 nm thick ITO film formed thereon was cleaned with an organic solvent, and then the ITO surface was cleaned by UV/ozone treatment. Then, the ITO electrode-equipped glass substrate was mounted within a vacuum deposition machine, and the pressure was reduced to 0.001 Pa or lower to form the transparent anode 2. Then, a film of HIM-1 represented by a structural formula indicated below was formed at a vapor deposition rate of 6 nm/min in a film thickness of 5 nm as the hole injection layer 3 so as to cover the transparent anode 2. On the hole injection layer 3, a film of the compound of Example 1 (Compound 7) was formed at a vapor deposition rate of 6 nm/min in a film thickness of 65 nm as the hole transport layer 4. On the hole transport layer 4, EMD-1 (NUBD370, produced by SFC Co., Ltd.) and EMH-1 (ABH113, produced by SFC Co., Ltd.) were binary vapor deposited at such vapor deposition rates that the vapor deposition rate ratio was EMD-1:EMH-1=5:95, whereby the luminous layer 5 was formed in a film thickness of 20 nm. On this luminous layer 5, ETM-1 represented by a structural formula indicated below and EIM-1 represented by a structural formula indicated below were binary vapor deposited at such vapor deposition rates that the vapor deposition rate ratio was ETM-1:EIM-1=50:50, whereby a film concurrently serving as the hole blocking layer 6 and the electron transport layer 7 was formed in a film thickness of 30 nm. On the hole blocking layer 6/electron transport layer 7, a film of EIM-1 was formed at a vapor deposition rate of 6 nm/min in a film thickness of 1 nm as the electron injection layer 8. Finally, aluminum was vapor deposited to a film thickness of 120 nm to form the cathode 9. The glass substrate having the organic films and the aluminum film formed thereon was moved into a glove box purged with dry nitrogen, and stuck to a sealing glass substrate by use of a UV curing resin to prepare an organic EL device. The resulting organic EL device was measured for the light emission characteristics when a direct current voltage was applied at normal temperature in the atmosphere. The results of the measurements are shown in Table 1.

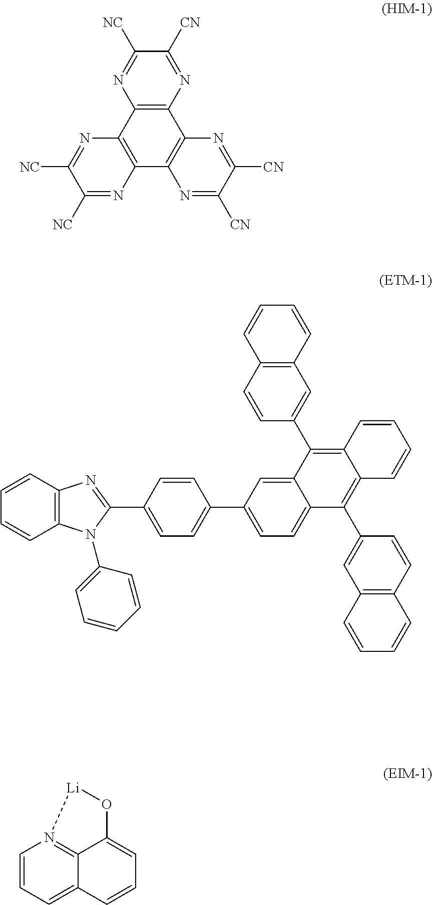

### Example 4

An organic EL device was prepared under the same conditions as in Example 3, except that the compound obtained in Example 2 (Compound 9) was used instead of the compound obtained in Example 1 (Compound 7) for the hole transport layer 4. The resulting organic EL device was measured for the light emission characteristics exhibited when a direct current voltage was applied at normal temperature in the atmosphere. The results of the measurements are shown in Table 1.

### Comparative Example 1

An organic EL device was prepared under the same conditions as in Example 3, except that HTM-A represented by the following structural formula was used instead of the compound obtained in Example 1 (Compound 7) for the hole transport layer 4. The resulting organic EL device was measured for the light emission characteristics exhibited when a direct current voltage was applied at normal temperature in the atmosphere. The results of the measurements are shown in Table 1.

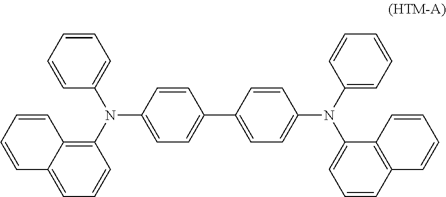 [SMILES: c1ccc(N(c2ccc(-c3ccc(N(c4ccccc4)c4cccc5ccccc45)cc3)cc2)c2cccc3ccccc23)cc1]

TABLE 1

| | | | | Luminous | Power |
|---|---|---|---|---|---|
| | | | Brightness | efficiency | efficiency |
| | | Voltage [V] | [cd/m²] | [cd/A] | [lm/W] |
| | Compound | (@10 mA/cm²) | (@10 mA/cm²) | (@10 mA/cm²) | (@10 mA/cm²) |
| Ex. 3 | Comp. 7 | 4.04 | 620 | 6.20 | 4.82 |
| Ex. 4 | Comp. 9 | 4.01 | 723 | 7.23 | 5.66 |
| Comp. Ex. 1 | HTM-4 | 4.08 | 516 | 5.16 | 3.97 |

As shown in Table 1, the driving voltage when an electric current at a current density of 10 mA/cm² was flowed showed values of 4.01 to 4.04V in the organic EL devices of the present invention. As compared with 4.08V in the organic EL device of Comparative Example 1, all the organic EL devices of the present invention were capable of low voltage driving. The power efficiency was 3.97 lm/W in the organic EL device of Comparative Example 1, while those in the organic EL devices of the present invention were 4.82 to 5.66 lm/W, showing great increases. In both of the brightness and the luminous efficiency, the organic EL devices of the present invention achieved improvements over the organic EL device of Comparative Example 1.

As noted above, the organic EL devices using the benzofuroindole derivatives of the present invention were found to be capable of achieving an increase in power efficiency and a decrease in practical driving voltage as compared with the known organic EL device using HTM-A.

### <Measurement of Light Emission Starting Voltage>

The light emission starting voltage (voltage when luminance reached 1 cd/m²) was measured. The results are shown below.

| Organic EL | | Light emission starting |
|---|---|---|
| device | Compound | voltage [V] |
| Example 3 | Compound 7 | 3.1 |
| Example 4 | Compound 9 | 3.1 |
| Comparative | HTM-A | 3.2 |
| Example 1 | | |

In comparison with Comparative Example 1 using HTM-A, Examples 3 and 4 were found to lower the light emission starting voltage.

As shown above, the organic EL devices of the present invention were excellent in the power efficiency, and were able to achieve decreases in the practical driving voltage, in comparison with devices using a general hole transport material (HTM-A).

### INDUSTRIAL APPLICABILITY

The benzofuroindole derivative of the present invention is high in hole transport capability, excellent in electron blocking capability, and stable in a thin film state, so that it excels as a material for an organic EL device. An organic EL device prepared using the benzofuroindole derivative of the present invention shows a high luminous efficiency and a high power efficiency, can lower practical driving voltage, and can improve durability. Thus, the organic EL device of the present invention can be put to uses such as domestic electrical appliances and illumination.

### Explanations of Letters or Numerals

1 Glass substrate
2 Transparent anode
3 Hole injection layer
4 Hole transport layer
5 Light emission layer
6 Hole blocking layer
7 Electron Transport layer
8 Electron injection layer
9 Cathode

1. A benzofuroindole derivative represented by the following general formula (1) 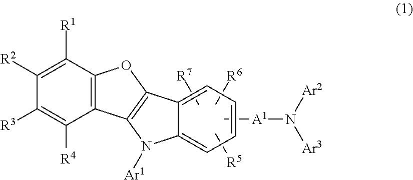
wherein,
Ar² and Ar³ may be the same or different, and each represent an aromatic hydrocarbon group, an aromatic heterocyclic group or a condensed polycyclic aromatic group,
R¹ to R⁷ may be the same or different, each represent a hydrogen atom, a deuterium atom, a fluorine atom, a chlorine atom, a cyano group, a nitro group, an alkyl group having 1 to 6 carbon atoms, a cycloalkyl group having 5 to 10 carbon atoms, an alkenyl group having 2 to 6 carbon atoms, an alkyloxy group having 1 to 6 carbon atoms, a cycloalkyloxy group having 5 to 10 carbon atoms, an aromatic hydrocarbon group, an aromatic heterocyclic group, a condensed polycyclic aromatic group or an aryloxy group, and may be bonded to each other via a single bond, a methylene group, an oxygen atom or a sulfur atom to form a ring,
A¹ represents a divalent group of an aromatic hydrocarbon, a divalent group of an aromatic heterocycle, a divalent group of a condensed polycyclic aromatic or a single bond,
Ar² and Ar³ may be bonded to each other via a single bond, a methylene group, an oxygen atom or a sulfur atom to form a ring, and
when A¹ is a divalent group of an aromatic hydrocarbon, a divalent group of an aromatic heterocycle or a divalent group of a condensed polycyclic aromatic, A¹ and Ar³ may be bonded to each other via a single bond, a methylene group, an oxygen atom or a sulfur atom to form a ring.

2. The benzofuroindole derivative according to claim 1, which is represented by the following general formula (2): 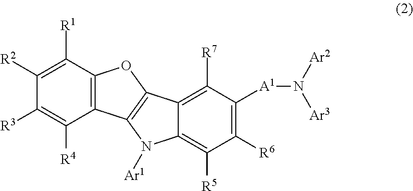
wherein,
Ar¹ to Ar³, R¹ to R⁷ and A¹ have the same meanings as those defined in the general formula (1).

3. The benzofuroindole derivative according to claim 1, wherein A¹ is a phenylene group.

4. An organic electroluminescent device comprising a pair of electrodes and at least one organic layer sandwiched therebetween, wherein the benzofuroindole derivative according to claim 1 is used as a constituent material for the at least one organic layer.

5. The organic electroluminescent device according to claim 4, wherein the organic layer is a hole transport layer.

6. The organic electroluminescent device according to claim 4, wherein the organic layer is an electron blocking layer.

7. The organic electroluminescent device according to claim 4, wherein the organic layer is a hole injection layer.

8. The organic electroluminescent device according to claim 4, wherein the organic layer is a luminous layer.
